(19) United States
(12) Patent Application Publication
(10) Pub. No.: US 2010/0048598 A1
(43) Pub. Date: Feb. 25, 2010

(54) TOPICAL COMPOSITIONS COMPRISING 5-ALPHA REDUCTASE INHIBITORS

(76) Inventors: Sateesh Kandavilli, Hyderabad (IN); Vijendra Nalamothu, Basking Ridge, NJ (US); Vishvabhavan Pandya, Sagar (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/544,737

(22) Filed: Aug. 20, 2009

Related U.S. Application Data
(60) Provisional application No. 61/106,733, filed on Oct. 20, 2008.

(30) Foreign Application Priority Data
Aug. 21, 2008 (IN) 2033/CHE/2008

(51) Int. Cl. A61K 31/506; A61K 31/473

(52) U.S. Cl. 514/275; 514/284

(57) ABSTRACT

The present invention relates to topical compositions comprising 5α-reductase inhibitors. The present invention also includes processes for preparation of such topical compositions and methods of using them.

### INTRODUCTION

Aspects of the present invention relate to topical compositions comprising 5α-reductase inhibitors. Further aspects include processes for preparation of such topical compositions and methods of using them.

5α-reductase is an enzyme that is responsible for conversion of testosterone, a male sex hormone, to dihydrotestosterone (DHT). DHT is an androgenic compound that causes hyperandrogenic conditions like enlargement of the prostate in men with progressing age, termed “benign prostatic hyperplasia.” Another consequence of increased DHT levels includes androgenic alopecia (AA), which is commonly termed “male pattern baldness.”

Androgenic alopecia is a common form of hair loss in both men and women. In men, particularly, this condition is also commonly known as male-pattern baldness. Hair is lost in a well-defined pattern, beginning above both temples. Over time, the hairline recedes to form a characteristic “M” shape. Hair also thins at the crown of the head, often progressing to partial or complete baldness. In women, the hair loss is all over the head and not patterned, and rarely leads to complete baldness. The etiology is mainly genetic and is clearly androgen-dependent. Therefore, 5α-reductase inhibitors that inhibit synthesis of DHT, can be useful in treating androgenic alopecia.

Currently, two treatments are approved by U.S. Food and Drug Administration for the treatment of AA in males: topical minoxidil and oral finasteride. While minoxidil (having a chemical name 6-piperidin-1-ylpyrimidine-2,4-diamine 3-oxide) is an arterial vasodilator, finasteride is a type II 5α-reductase inhibitor. Finasteride, at the dose of 1 mg/day, slowed the progression of hair loss and increased hair growth in treated men compared with those in a control group. (K. Kaufman et. al., Journal of the American Academy of Dermatology, Vol. 39, pages 578-589, 1998; and J. Leyden et al., Journal of the American Academy of Dermatology, Vol. 40, pages 930-937, 1999). In the case of finasteride, with a daily oral 1 mg dose, scalp DHT is reduced by 64%, and serum DHT by 68% (J. Drake et al., Journal of the American Academy of Dermatology, Vol. 44, pages 550-554, 1999).

One compound in the 5α-reductase inhibitor class is dutasteride. Dutasteride has a chemical name (5α,17β)-N-{2,5-bis(trifluoromethyl)phenyl}-3-oxo-4-azaandrost-1-ene-17-carboxamide. It has the structural formula shown as Formula I.

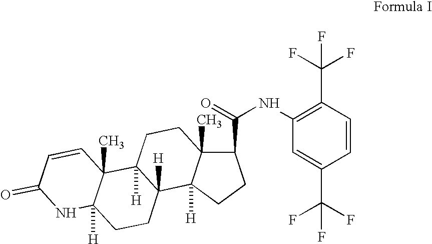

Dutasteride is a synthetic 4-azasteroid compound that is a selective inhibitor of both type I and type II isoforms of steroid 5α-reductase (5AR), an intracellular enzyme that converts testosterone to 5α-dihydrotestosterone (DHT), and is indicated for the treatment of symptomatic benign prostatic hyperplasia (BPH) in men. A pharmaceutical product containing dutasteride as the active ingredient is commercially available as AVODART® from GlaxoSmithKline, in the form of soft gelatin capsules for oral administration and containing 0.5 mg of the active ingredient.

R. Clerk et al., Journal of Clinical Endocrinology and Metabolism, Vol. 89, pages 2179-2184, 2004, demonstrated approximately 3 times higher potency of dutasteride at inhibiting type II 5α-reductase and more than 100 times higher potency at inhibiting the type I isoform, as compared with finasteride.

While type I 5α-reductase has been reported to be located predominantly in the skin, both in hair follicles and sebaceous glands, as well as in the liver, prostate, and kidney, type II 5α-reductase is found in the male genitalia, prostate and hair follicles. Due to the distribution of both isoforms of 5α-reductase in skin, it has been hypothesized that dutasteride, being a dual inhibitor, would be more useful for treatment of AA when compared to the selective type 11 inhibitor such as finasteride. (S. Marihart et. al., Reviews in Urology, Vol. 7, pages 203-210, 2005).

Dutasteride is a highly lipophilic molecule (Log P=6.8). It is insoluble in water, soluble in ethanol, methanol and polyethylene glycol 400. Absolute bioavailability of orally administered dutasteride is only about 60% (40%-94%). Some of the known approaches to improve solubility characteristics and bioavailability of drug compounds include salt formation, particle size reduction, pH adjustment, use of surfactants, inclusion complexes with cyclodextrins, use of oily formulations, use of self-emulsifying drug delivery systems, formation of co-precipitates with hydrophilic polymers, and co-milling with hydrophilic excipients, to name a few.

U.S. Pat. No. 5,565,467 discloses dutasteride, pharmaceutical compositions comprising dutasteride and their use in the treatment of benign prostatic hyperplasia. U.S. Pat. Nos. 5,846,976 and 5,998,427 disclose methods of treating an androgen-responsive or androgen-mediated condition in a mammal, azasteroids and dutasteride compounds, and a method of inhibiting testosterone 5α-reductase, respectively. U.S. Pat. No. 7,022,854 describes two polymorphic forms of dutasteride: crystalline Form II and an amorphous form, and processes of preparation of crystalline Form I, Form II and the amorphous form. International Application Publication No. WO 2007/120263 discloses preparation of dutasteride having low amounts of impurities, and a process of purifying dutasteride.

U.S. Pat. No. 6,998,138 and U.S. Patent Application Publication No. 2004/0096405 disclose topical and transdermal formulations for treatment of androgenic alopecia. U.S. Pat. Nos. 6,818,226 and 6,299,900, and U.S. Patent Application Publication Nos. 2004/0146469, 2007/0071803 and 2007/0255197, disclose compositions containing dermal penetration enhancers.

Aspects of the present invention address the need of improving delivery of 5α-reductase inhibitors such as dutasteride. In an aspect, the present invention provides pharmaceutical compositions for topical delivery of 5α-reductase inhibitors. The compositions provide enhanced local and/or systemic delivery of the 5α-reductase inhibitors. Aspects of the present invention also provide processes for making the compositions and methods of using such pharmaceutical compositions.

### SUMMARY

An aspect of the present invention provides pharmaceutical compositions for improved topical delivery of 5α-reductase inhibitors.

In another aspect, the present invention provides pharmaceutical compositions for improved topical delivery of dutasteride, including salts, esters, isomers, solvates, hydrates, and polymorphs thereof.

In another aspect, compositions are provided comprising at least one 5α-reductase inhibitor active agent, such as dutasteride, to deliver the active agent onto the skin, onto the scalp, beneath the surface of the skin and/or scalp, and/or into the systemic circulation.

In yet another aspect, compositions of present invention comprise a 5α-reductase inhibitor as an active agent and at least one pharmaceutically acceptable carrier, and optionally one or more other pharmaceutically acceptable excipients.

In another aspect, the pharmaceutically acceptable carriers for use in the present invention are hydrophilic, hydrophobic, lipophilic, or ampiphilic, or mixtures thereof.

In one aspect, pharmaceutical compositions of the present invention are in the form of “permeation enhanced” compositions, or “penetration enhanced” compositions, or “depot” compositions.

In yet another aspect, pharmaceutical compositions of the present invention are in the form of solutions, ointments, creams, gels, lotions, suspensions, mousses, aerosols, sprays, foams, microspheres, microemulsions, nanoemulsions, nanoparticles, nanosuspensions, dermal sticks, roll-ons, pumps, patches, tapes, or the like.

In an embodiment, pharmaceutical compositions of the present invention exhibit excellent physicochemical stability during storage at conditions of 40° C. and 75% relative humidity (RH) over a period of at least 6 months.

In an embodiment, pharmaceutical compositions of the present invention include less than about 2%, less than about 1%, or less than about 0.5%, of total impurities, based on the label content of a contained 5α-reductase inhibitor, after storage at 40° C. and 75% RH for at least 6 months.

In an embodiment, pharmaceutical compositions of the present invention have a viscosity of about 0.5 poise to about 3 poise, or about 1 poise to about 2.5 poise, after storage at 40° C. and 75% RH for at least 6 months.

In an embodiment, pharmaceutical compositions of the present invention have pH values less than about 7 and greater than about 1, or less than about 5.5 and greater than about 2.5, after storage at 40° C. and 75% RH for at least 6 months.

In an aspect, the present invention provides processes for the preparation of pharmaceutical compositions for improved topical delivery of 5α-reductase inhibitors.

In an aspect, the present invention provides processes for the preparation of pharmaceutical compositions for improved transdermal delivery of 5α-reductase inhibitors.

In another aspect, the present invention provides methods of using pharmaceutical compositions described herein, for the prophylaxis, amelioration, and/or treatment of androgenic alopecia.

In another aspect, pharmaceutical compositions of the present invention comprise at least one 5α-reductase inhibitor as an active agent, which is useful in the prophylaxis, amelioration or treatment of androgenic alopecia, and additionally comprise at least one another active agent.

### DETAILED DESCRIPTION

Since the hair follicles in the skin are potential sites of action for dutasteride, formulations of dutasteride that can be applied to the skin will enhance the local bioavailability of dutasteride at hair follicles.

Structurally, skin consists of two major parts: the ‘epidermis’ (outermost layer) and the ‘dermis’ (thicker inner region). The outermost layer of the epidermis is the ‘stratum corneum’ that consists of flattened dead cells which are filled with keratin. The regions between flattened dead cells of the stratum corneum are filled with lipids which form a lamellar phase that is responsible for natural barrier properties of the skin. Topical formulations can enhance the local drug bioavailability by permeation through the skin, while a transdermal formulation would provide prolonged systemic levels of the drug.

For effective delivery on the skin surface, an active agent must be partitioned from the formulation vehicle into the stratum corneum, and then must be diffused within the stratum corneum. Later, the drug must be partitioned to local tissues including epidermis, dermis, subcutis and appendageal for topical delivery, and/or into epidermis, dermis and bloodstream for effective transdermal delivery. Dermal penetration enhancers may be used to facilitate transport across the dermal layer.

Aspects of the present invention provide pharmaceutical compositions for improved topical delivery of 5α-reductase inhibitors. A 5α-reductase inhibitor according to the present invention includes finasteride, dutasteride, or any other 5α-reductase type I or type II inhibitor. In embodiments, the present invention provides pharmaceutical compositions for improved topical delivery of dutasteride, including salts, esters, isomers, solvates, hydrates and polymorphs thereof.

Embodiments of compositions of the present invention comprise a 5α-reductase inhibitor as an active agent and at least one pharmaceutically acceptable carrier, optionally together with one or more other pharmaceutically acceptable excipients. Pharmaceutically acceptable carriers of the present invention are hydrophilic, hydrophobic, lipophilic, or ampiphilic.

The active agent dutasteride according to the present invention can be present in an amorphous form, or in any crystalline form such as Form I or II, or mixtures thereof. Further, embodiments of pharmaceutical formulations of the present invention comprise an active agent together with a pharmaceutically acceptable carrier, optionally together with one or more other pharmaceutically acceptable excipients, in the form of a co-precipitate, premix, solid dispersion, solution, solubilized system, co-crystals, or the like.

Compositions of the present invention comprising at least one 5α-reductase inhibitor(s) such as dutasteride are intended to deliver the active agent onto the skin and/or scalp, and/or just beneath the skin and/or scalp, and/or into systemic circulation.

The terms “drug” or “active agent” are used interchangeably, and describe a pharmacologically active substance that is present in the composition in a quantity that is sufficient to elicit an intended pharmacological response.

Certain pharmaceutical compositions of the present invention are in the form of “permeation enhanced” compositions, or “penetration enhanced” compositions, or “depot” compositions.

The term “pharmaceutical composition” as used herein refers to compositions comprising one or more 5α-reductase inhibitors together with one or more pharmaceutically acceptable excipients as required to prepare a dosage form for the effective delivery of the active agent. Such pharmaceutical compositions can be in the form of solutions, ointments, creams, gels, lotions, suspensions, sprays, foams, microspheres, microemulsions, nanoemulsions, nanoparticles, nanosuspensions, dermal sticks, roll-ons, pumps, patches, tapes, and the like.

The term “topical composition” means a composition that is applied onto the skin surface. Such “topical” composition may act ‘locally’ or ‘transdermally’. A “transdermal” composition refers to a composition that can be applied to the body surface, which then may permeate through the stratum corneum or scalp to form a reservoir just beneath the skin surface, or may be absorbed systemically to provide desirable drug levels in the circulation.

“Skin depot” or “depot” refers to a pharmaceutical composition that provides storage of drug within the skin and releases the contained drug to surrounding tissue over a prolonged period of time, and/or delayed to commence after a period of time.

“Scalp” according to the present invention means the skin covering the head and is bordered by the face anteriorly and the neck to the sides and posteriorly.

“Penetration enhancement” or “permeation enhancement” means an increase in the permeability of a biological membrane (i.e. skin or mucosa) to a drug, so as to increase the rate at which the drug is transported through the membrane. “Permeation enhancer”, “enhancer”, “penetration enhancer”, or similar terms mean a material that achieves such permeation or penetration enhancement, and an “effective amount” of an enhancer means an amount effective to enhance penetration through the skin or mucosa of a selected agent to a desired degree.

Suitable carriers according to the present invention include, but are not limited to: paraffin oils; esters of C₈-C₁₈ organic acids like isopropyl myristate; C₈-C₃₀ fatty alcohols; silicone oils; vegetable oils; fractionated or hydrogenated vegetable oils; monoglycerides; diglycerides; triglycerides; phospholipids; dimethyl isosorbide; volatile solvents; N-methylpyrrolidone; N,N-dimethylacetamide and N,N-dimethylformamide; dimethylsulphoxide; alcohols such as ethanol and isopropyl alcohol; glycols such as propylene glycol, polyethylene glycol and glycerol; cyclodextrins such as beta-cyclodextrin, beta-hydroxy cyclodextrin, gamma-cyclodextrin, and hydroxypropyl cyclodextrin; and any mixtures thereof.

Suitable other pharmaceutically acceptable excipients include, but are not limited to, surfactants, co-surfactants, penetration enhancers, antioxidants, buffering agents, preservatives, viscosity modifying agents, chelating/complexing agents, coloring agents, perfumes, polymers, gelling agents, alcohols, liquid or semi-solid oily components, and any mixtures thereof.

The other excipients that are useful in the pharmaceutical compositions of the present invention can serve more than one purpose, such as, for example, a surfactant or co-surfactant used in the present invention can also act as a penetration or permeation enhancer.

In an aspect, pharmaceutical compositions of the present invention comprise at least one 5α-reductase inhibitor as an active agent, one or more volatile solvents, one or more surfactants, optionally at least one penetration enhancer, and optionally one or more other pharmaceutically acceptable excipients.

According to an aspect of the present invention, the drug is dissolved or dispersed in a suitable pharmaceutically acceptable carrier. For example, when a composition of the present invention is in the form of a dispersion, it comprises at least one surfactant and optionally one or more co-surfactants along with a pharmaceutically acceptable carrier. A surfactant may be an anionic, cationic, non-ionic, or zwitterionic.

Suitable surfactants useful in the present invention include, without limitation, sodium laurate, sodium stearate, sodium lauryl sulfate, cetyl trimethyl ammonium bromide, benzalkonium chloride, a poloxamer (e.g., 231, 182, and 184), a polyoxyethylene sorbitan ester (e.g., Tween™ 20, 40, 60, and 80), lecithin, and any mixtures thereof.

Other suitable surfactants include glycerol fatty acid esters such as glycerol monostearate, glycol fatty acid esters such as propylene glycol monostearate, polyhydric alcohol fatty acid esters such as polyethylene glycol (400) monooleate, polyoxyethylene fatty acid esters such as polyoxyethylene (40) stearate, polyoxyethylene fatty alcohol ethers such as polyoxyethylene (20) stearyl ether, polyoxyethylene sorbitan fatty acid esters such as polyoxyethylene sorbitan monostearate, sorbitan esters such as sorbitan monostearate, alkyl glycosides such as cetearyl glucoside, sulfated oils such as a sulfuric ester of ricinoleic acid disodium salt, and sulfonated compounds such as alkyl sulfonates including sodium cetane sulfonate, amide sulfonates such as sodium N-methyl-N-oleyl laurate, sulfonated dibasic acid esters such as sodium dioctyl sulfosuccinate, alkyl aryl sulfonates such as sodium dodecylbenzene sulfonate, alkyl naphthalene sulfonates such a sodium isopropyl naphthalene sulfonate, a petroleum sulfonate such as aryl naphthalene with alkyl substitutes. Examples of suitable cationic surfactants include amine salts such as octadecyl ammonium chloride.

Suitable penetration enhancers that can be used in the present invention include, but are not limited to: sulfoxides such as dimethylsulfoxide (DMSO) and decylmethylsulfoxide (C₁₀ MSO); ethers such as diethylene glycol monoethyl ether (available commercially as Transcutol™) and diethylene glycol monomethyl ether; 1-substituted azacycloheptan-2-ones, such as 1-n-dodecyl-cyclazacycloheptan-2-one; alcohols such as propanol, octanol, benzyl alcohol, and the like; fatty acids such as lauric acid, oleic acid, and valeric acid; fatty acid esters such as isopropyl myristate, isopropyl palmitate, methylpropionate, and ethyl oleate; polyol esters such as butanediol and polyethylene glycol monolaurate, amides and other nitrogenous compounds such as urea, N,N-dimethylacetamide (DMA), N,N-dimethylformamide (DMF), 2-pyrrolidone, 1-methyl-2-pyrrolidone, ethanolamine, diethanolamine, and triethanolamine; terpenes and terpinoids; alkanones; organic acids, such as salicylic acid and salicylates, citric acid and succinic-acid and the like; and any mixtures thereof.

Antioxidants that are useful in the present invention include, but are not limited to, tocopherol succinate, ascorbic acid, propyl gallate, vitamin E, butylated hydroxytoluene, butylated hydroxyanisole, sodium pyrosulfite, kojic acid, cysteine, hydroquinone, and the like, including any mixtures thereof.

Buffering agents that are useful in the present invention include, but are not limited to: alkali metal salts such as potassium and sodium carbonates, acetates, borates, phosphates, citrates and hydroxides; weak acids such as acetic, boric and phosphoric acids, and the like; and mixtures thereof.

Preservatives that are useful in the present invention include, but are not limited to, diazolidinyl urea and imidazolidinyl urea, as well as methyl, ethyl, propyl and butyl esters of p-hydroxybenzoic acid (parabens), isothiazolones, and the like, including any mixtures thereof.

Viscosity modifying agents that are useful in the present invention include, but are not limited to, cetyl alcohol, glycerol, polyethylene glycol (PEG), PEG-stearate, xanthan gums such as Keltrol™, and the like, including any mixtures thereof.

Chelating or complexing agents that are useful in the present invention include but are not limited to ethylenediaminetetraacetic acid (EDTA) and its derivatives, thioglycolic acid, thiolactic acid, thioglycerol, and the like, including mixtures thereof.

Polymers that are useful in the present invention include, but are not limited to, bioadhesive agents, gelling agents, film-forming agents, phase change agents, and any mixtures thereof. Examples of polymers include, but are not limited to, ethylcelluloses, acrylates, methacrylates, pyrrolidone polymers, including polymers of N-vinylpyrrolidone, polyoxyethylenes, hydroxypropyl methylcelluloses, hydroxypropyl celluloses, polymethylmethacrylates, cellulose acetates and their derivatives, cellulose acetate phthalates, hydroxypropyl methylcellulose phthalates, shellac, methacrylic acid based polymers such as those sold under the tradename EUDRAGIT™, zein, polycarbonates, polyorthoesters, polydioxanones, polyacetals, polyhydroxybutyrates, polyhydroxy valerates, polyethers, polyphosphazenes, polyhydroxy celluloses, polyalkylene oxalates, polyorthocarbonates, polyphosphoesters, star-branched polymers and copolymers, polysaccharides, polyketals, polyalkylene succinates, polypropylene oxides, chitin, chitosan, and other polymers known to a person skilled in the art of drug delivery, including copolymers, terpolymers, combinations and the likes, and any mixtures thereof.

Gelling agents that are useful in the present invention include, but are not limited to, cellulose and its derivatives, such as sodium carboxymethyl celluloses and hydroxyalkyl and alkyl celluloses, carbomers such as Carbopol™ and their derivatives, carob, carregeenans and derivatives, xanthan gum, sclerane gum, long chain alkanolamides, bentone and derivatives, kaolin, green clay, bentonite, magnesium aluminum silicate (Veegum™), guar gums (such as Jaguar™ HP-120), cross-linked acrylic acid polymers, and the like, including any mixtures thereof.

Liquid oily components that are useful in the present invention include, but are not limited to, sunflower oil, soybean oil, peanut oil, canola oil, cottonseed oil, coconut oil, palm oil, palm kernel oil, corn oil, flax seed oil, olive oil, safflower oil, fish oil, liquid state triglyceride esters of fatty acids, and the like, including any mixtures thereof.

Semi-solid or solid oily components that are useful in the present invention include, but are not limited to, C₁₂-C₃₀ higher fatty acids, e.g., stearic acid and linoleic acid, solid state mono-, di- and tri-glyceride esters of fatty acids, higher saturated alcohols, including aliphatic alcohols having 14-30 carbon atoms such as cetostearyl alcohol, waxes, such as carnauba wax, hydrocarbons, such as soft and hard paraffins, sphingolipids, and the like, including any mixtures thereof.

In embodiments of the present invention, pharmaceutical compositions provide topical delivery of 5α-reductase inhibitors such as dutasteride to enhance the availability of the active agent to the hair follicles in the scalp, particularly when applied onto the scalp.

In embodiments, pharmaceutical compositions of the present invention, upon administration, permit the drug to penetrate through the skin or the scalp and thereby provide pharmacologically effective systemic drug levels.

In embodiments, compositions of the present invention form a depot or a reservoir at or near the point of application, and exhibit a pharmacological effect for an extended duration of time, and/or commencing after a delayed time, after application.

Further, compositions of the present invention are easy to formulate and frequently are removable by water washing. Also, in aspects, the compositions of the present invention have appreciable spreadability and can be easily applied to the skin.

Pharmaceutical compositions of the present invention comprising at least one 5α-reductase inhibitor as an active agent, can additionally comprise at least one another active agent. Such other active agents can either enhance or potentiate the activity of a 5α-reductase inhibitor or are useful for management (prophylaxis, amelioration or treatment) of any associated diseases/disorders, for which said 5α-reductase inhibitors are indicated. In certain embodiments, such additional active agents may be chemical compounds or extracts of one or more active components obtained form a natural source, such as plant extracts.

Such additional active agents include but are not limited to: hair loss preventing agents; hair growth promoting agents; anti-alopecia agents such as finasteride, FCE 28260, and minoxidil; anti-infectives; antibacterials; antifungals; antihistaminics; immunomodulatory agents; anti-dandruff agents; antivirals; antiandrogenic agents such as fluconazole, ketoconazole and spironolactone; hormones; steroids; and the like.

In embodiments, topical pharmaceutical compositions according to the present invention provide improved systemic delivery of an active agent, i.e., a 5α-reductase inhibitor, such as dutasteride, that results in enhanced bioavailability and is substantially comparable in effect to an oral or injectable formulation of the 5α-reductase inhibitor.

In embodiments, the rate of diffusion of the drug into the skin is such that therapeutic concentrations of the drug are achieved within about 0.5-8 hours after topical administration of the composition, and a desirable drug level is maintained for an extended duration of time, such as for about 4-24 hours.

The dose of the active agent, i.e., the 5α-reductase inhibitor, administered topically, particularly depends on the severity of the disorder, the history of the subject and the dosage form. Typically, a dose is in the range of about 0.1 mg/day to about 900 mg/day, administered once-daily, twice-daily, three times per day, or four times per day.

In an embodiment the pharmaceutical composition of the present invention contains less than about 1% or less than about 0.5% of the impurities shown in Formulae II-VI, based on the label drug content.

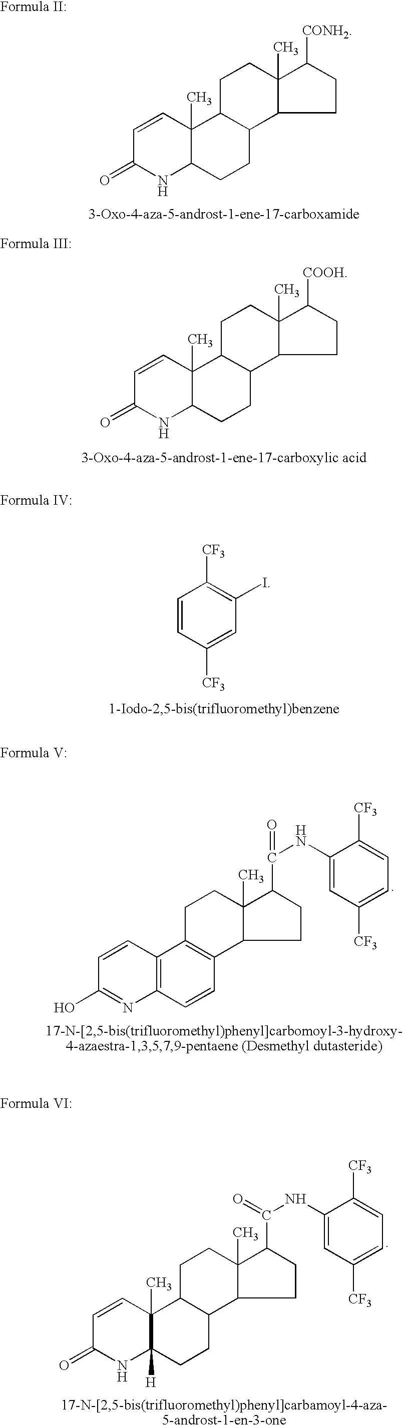

In one embodiment, the impurities in the pharmaceutical composition may be incorporated from one or more of the sources including the starting material of dutasteride synthesis, impurities related to process of synthesis like intermediate and other products; and degradation impurities developed during storage of the pharmaceutical composition.

In embodiments, pharmaceutical compositions of the present invention contain less than about 1%, or less than about 0.5%, based on the label drug content, of total impurities represented by Formulae II-VI.

The impurities can be determined using a high performance liquid chromatographic method of analysis with gradient elution. The instrument specifications and chromatographic conditions are described as follows.

| | |
|---|---|
| Detector: | Variable length UV absorbance detector. |
| Column: | Zorbax SB C18 250 × 4.6 mm, 5 μm or equivalent. |
| Mobile phase (A): | Potassium phosphate buffer, pH 3.0. |
| Mobile phase (B): | Acetonitrile-water (90:10 by volume). |
| Flow rate: | 1 mL/minute. |

In embodiments, pharmaceutical compositions of the present invention exhibit excellent physicochemical stability after storage at 40° C. and 75% RH for at least 6 months.

In embodiments, pharmaceutical compositions of the present invention have less than about 2%, or less than about 1%, or less than about 0.5%, based on the label drug content, of total impurities represented by Formulae II-VI, after storage in closed containers at 40° C. and 75% RH for at least 6 months.

In embodiments, pharmaceutical compositions of the present invention have viscosities of about 0.5 poise to about 3 poise, or about 1 poise to about 2.5 poise, after storage in closed containers at 40° C. and 75% RH for at least 6 months. The viscosity was determined using Brookfield viscometer CAP 2000+model at 25° C.

In embodiments, pharmaceutical compositions of the present invention have pH values less than about 7 and more than about 1, or less than about 5.5 and more than about 2.5, after storage in closed containers at 40° C. and 75% RH for at least 6 months.

The compositions of the present invention are safe, effective and well-tolerated, and are useful for the management, such as prophylaxis, amelioration and/or treatment, of 5α-reductase inhibitor indicated diseases/disorders, such as for use in treating androgenic alopecia.

In embodiments, compositions of the present invention are stable both during the production process and during storage for commercially relevant times, e.g., for six months, 1 year, 2 years, or 3 years, in closed packages.

Aspects of the present invention provide methods for preparing pharmaceutical compositions of the present invention. In an embodiment, a process for preparation of a composition of the present invention comprises combining the active agent 5α-reductase inhibitor with at least one pharmaceutically acceptable carrier, optionally with one or more other pharmaceutically acceptable excipients, and formulating into a suitable topical dosage form.

In an embodiment, a method of preparing a pharmaceutical composition of the present invention comprises:

(a) dissolving or dispersing an active agent in a carrier, which is hydrophilic, hydrophobic or both;

(b) adding a dermal penetration enhancer;

(c) optionally, adding one or more surfactants;

(d) optionally, adding one or more pharmaceutically acceptable excipients; and

(e) forming the mixture into a gel or cream.

In embodiments, a method of preparation of the pharmaceutical composition includes use of a solution or dispersion of the drug in a hydrophobic vehicle.

In embodiments, a method of preparation of the pharmaceutical compositions includes use of a solution or dispersion of the drug in a hydrophilic vehicle.

The methods of manufacturing of the present invention may include filling compositions of the present invention into appropriate containers. The compositions of the present invention may be packaged, for example, into unit dose or multi-dose containers. The inner surface of the container may further comprise a liner or be treated to protect the container surface and/or to protect the composition from adverse affects that may arise from the composition being in contact with the inner surface of the container. Examples of liners or coating materials include, but are not limited to, high density polyethylene, low density polyethylene, very low density polyethylene, polyethylene copolymers, thermoplastic elastomers, silicon elastomers, polyurethane, polypropylene, polyethylene terephthalate, nylon, flexible polyvinylchloride, natural rubber, synthetic rubber, and combinations thereof. Liners or coating material are typically substantially impermeable to the composition and typically to the individual components of the composition.

In embodiments, the invention includes the use of packaging materials such as containers and closures of high-density polyethylene (HDPE), low-density polyethylene (LDPE) and/or polypropylene and/or glass, collapsible aluminum foil, etc. The packages containing the formulations of the present invention can optionally contain a desiccant, oxygen absorbent, or both. In embodiments, the packages according to the present invention may be flushed with nitrogen or other inert gases, before fitting with a closure.

The compositions of the present invention may be applied to a skin surface or a mucosal membrane using a variety of means, including, but not limited to a pump-package, a brush, a swab, a finger, a hand, or other applicator.

In embodiments, the pharmaceutical compositions of the present invention comprising at least one 5α-reductase inhibitor as an active agent, optionally with another active agent, are useful in the prophylaxis, amelioration or treatment of androgenic alopecia. In an aspect, the present invention provides methods of using pharmaceutical compositions according to the present invention, such as in the management (prophylaxis, amelioration and/or treatment) of androgenic alopecia, and optionally one or more associated diseases/disorders thereof.

An embodiment of a method of prophylaxis, amelioration or treatment using a composition of the present invention comprises administering to a subject in need thereof a prophylactically or therapeutically effective amount of the composition.

In embodiments, the compositions of the present invention are non-staining and non-irritating at the site of application. Further, the compositions of the present invention, in embodiments, provide a zero order, pseudo-zero order, first order, or pseudo-first order release of a drug.

The following examples further describe certain specific aspects and embodiments, are provided solely for purposes of illustration, and should not be construed as limiting the scope of the invention in any manner.

### Example 1

### Gel Composition Containing Dutasteride

| Ingredient | Percent (w/v) |
|---|---|
| Dimethylisosorbide | 15 |
| Propylene glycol | 25 |
| Ethanol | 45 |
| Dutasteride | 0.5 |
| Hydroxypropyl cellulose | 1.5 |
| Water | 15 |

Manufacturing Process:

1. Dimethylisosorbide, about 80% of the propylene glycol and about ⅔ of the ethanol are measured into a container.

2. In another container, dutasteride is added to the remaining portions of propylene glycol and ethanol, and mixed to produce a clear solution.

3. The mixture of step 1 is added to the solution of step 2, and stirred to obtain a clear solution.

4. Water is added with stirring.

5. Hydroxypropyl cellulose is dispersed into the solution under vigorous stirring and allowed to swell completely under slow stirring.

### Example 2

### Microemulsion in Gel Composition Containing Dutasteride

| Ingredient | Percent (w/v) |
|---|---|
| Dimethylisosorbide | 15 |
| Propylene glycol | 20 |
| Ethanol | 45 |
| Dutasteride | 0.5 |
| Oleic acid | 5 |
| Hydroxypropyl methylcellulose | 1.5 |
| Water | 15 |

Manufacturing Process:

1. Dimethylisosorbide, propylene glycol and ⅔ of the ethanol are measured into a container.

2. In another container, dutasteride is added to oleic acid and the remaining portion of ethanol, and mixed to produce a clear solution.

3. The solvent mixture of step 1 is added to the solution of step 2, and stirred to obtain a clear solution.

4. Water is added to the solution of step 3 under stirring.

5. Hydroxypropyl cellulose is dispersed in the solution of step 4 under vigorous stirring and is allowed to swell completely under slow stirring, to form a gel.

### Example 3

### Gel Composition Containing Finasteride

| Ingredient | Percent (w/v) |
|---|---|
| Dimethylisosorbide | 15 |
| Propylene glycol | 20 |
| Ethanol | 45 |
| Dutasteride | 0.5 |
| Transcutol ®* | 5 |
| Hydroxypropyl methylcellulose | 1.5 |
| Water | 15 |

*Transcutol ® is diethylene glycol monoethyl ether, from Gattafosse.

Manufacturing Process:

1. Dimethylisosorbide, propylene glycol and ⅔ of the ethanol are measured into a container.

2. In another container, finasteride is added to Transcutol and the remaining portion of ethanol, and mixed to produce a clear solution.

3. The solvent mixture of step 1 is added to the solution of step 2, and stirred to obtain a clear solution.

4. Water is added to the solution of step 3 under stirring.

5. Hydroxypropyl methylcellulose is dispersed in the solution of step 4 under vigorous stirring and allowed to swell completely under slow stirring, to form a gel.

### Example 4

### Dermal Stick Composition Containing Dutasteride

| Ingredient | Percent (w/v) |
|---|---|
| Ceresine wax | 14.9 |
| Cetyl alcohol | 14 |
| Castor oil | 15 |
| Glyceryl stearate | 8 |
| Dimethylisosorbide | 7.5 |
| Propylene glycol | 10 |
| Ethanol | 15 |
| Oleic acid | 3 |
| Dutasteride | 5 |
| Water | 7.6 |

Manufacturing Process:

1. Ceresine wax, cetyl alcohol, castor oil, and glyceryl stearate are melted at 65-75° C.

2. Dimethylisosorbide, propylene glycol, ethanol, oleic acid and water are mixed in a separate vessel and warmed to 50° C.

3. Dutasteride is added to the mixture of step 2 and stirred to dissolve the drug completely.

4. The aqueous phase of step 3 is added to the oil phase of step 1 under stirring, and the mixture is filled into a stick mold at about 50° C.

5. The mass is allowed to cool to about 25° C. and solidify in the mold, under controlled conditions, and the solidified mass is allowed to cure for 24 hours at about 25° C.

### Example 5

### Ointment Composition Containing Dutasteride

| Ingredient | Wt. Percent |
|---|---|
| Liquid paraffin | 5 |
| White paraffin | 10 |
| Glycerin | 30 |
| Cetosteryl alcohol | 8 |
| Methyl paraben | 0.3 |
| Polyoxyethylene sorbitan monostearate (Tween ™ 60) | 3.6 |
| Glyceryl monostearate | 2 |
| Dutasteride | 0.5 |
| Liquid paraffin | 5 |
| White beeswax | 5 |
| Water | q.s. to 100 |

Manufacturing Process:

1. Water, Tween 60, and glycerin are mixed with agitation, and the mixture is heated to 61-65° C.

2. Methyl paraben is dissolved in the solution of step 1 while maintaining the temperature, to form an aqueous phase.

3. Liquid paraffin, cetostearyl alcohol, white petrolatum, glyceryl monostearate, and white beeswax are mixed together and heated to 71-75° C., to form an oil phase.

4. Dutasteride is dispersed into the oil phase.

5. The oil phase of step 4 is added with stirring to the aqueous phase of step 2 at 61-65° C., and mixed by stirring.

6. The mixture is allowed to cool to room temperature under vacuum, to form an ointment.

### Example 6

### Cream Composition Containing Finasteride

| Ingredient | Wt. Percent |
|---|---|
| Finasteride | 1 |
| Propylene glycol | 6 |
| Dimethylisosorbide | 15 |
| Liquid paraffin | 0.5 |
| Cetomacrogol 1000* | 2 |
| Cetosteryl alcohol | 8 |
| White soft paraffin | 18 |
| Ethanol | 10 |
| Propyl paraben | 0.035 |
| Methyl paraben | 0.1 |
| Water | q.s. to 100 |

*Cetomacrogol 1000 is a macrogol ether containing 20 to 24 oxyethylene

groups, used as a solubilizer and emulsifier.

Manufacturing process.

1. Liquid paraffin, cetosteryl alcohol, white soft paraffin and half the Cetomacrogol 1000 are mixed in a clean and dry vessel and heated to 70-75° C. with stirring, while maintaining temperature in the range of 65-70° C., to form an oil phase.

2. In another vessel, propyl paraben and methyl paraben are dissolved in water at 90° C. and allowed to cool to 65-70° C., to form an aqueous phase.

3. Dimethylisosorbide, remaining cetomacrogol 1000, and ethanol are added to the aqueous phase of step 2, and dissolved by mixing for 10-15 minutes at 65-70° C.

4. The oil phase from step 1 is added to the aqueous phase of step 3 under vacuum transfer, while stirring slowly at about 60° C., then homogenized at about 60° C. and a vacuum of 0.4 bar, and the mixture is cooled.

5. In a separate vessel, finasteride and propylene glycol are mixed and stirred at 45° C. using a homogenizer to make a slurry.

6. The slurry of step 5 is added to the mixture of step 4 at 45° C. and mixed for 10 minutes at 9500 RPM to form a smooth cream.

7. The cream is cooled to 30° C. under a vacuum of 0.4 bar, while stirring, and is filled into collapsible tubes.

### Example 7

### Lotion Composition Containing Dutasteride

| Ingredient | Wt. Percent |
|---|---|
| Dutasteride | 0.5 |
| Minoxidil | 2.5 |
| Dimethylisosorbide | 15 |
| Methyl paraben | 0.15 |
| Propyl paraben | 0.03 |
| Propylene glycol | 25 |
| Hydroxypropyl cellulose | 0.5 |
| Ethanol | 41.5 |
| Water | q.s. to 100 |

Manufacturing Process:

1. Ethanol, propylene glycol, and dimethylisosorbide are mixed in a clean and dry vessel; and dutasteride and minoxidil are added and mixed to produce a clear solution.

2. Hydroxypropyl cellulose is added to the drug solution of step 1, in small portions under stirring.

3. Methyl paraben and propyl paraben are dissolved in water, and then added to material of step 2 with vigorous stirring.

4. The stirring is continued overnight to allow the viscosity to stabilize and then the lotion is filled into suitable containers.

### Example 8

### Gel Composition Containing Minoxidil

| Ingredient | Wt. Percent |
|---|---|
| Minoxidil | 5 |
| Disodium EDTA | 0.05 |
| Propylene glycol | 22 |
| Sorbic acid | 0.1 |
| Dimethylisosorbide | 15 |
| Carbopol ™ 980* | 2.5 |
| Benzyl alcohol | 0.5 |
| Cyclomethicone and dimethicone copolyol | 2 |
| PPG-20 methyl glucan ether distearate | 2 |
| Triethanolamine | 0.8 |
| Ethanol | 40 |
| Water | q.s. to 100 |

*Carbopol 980 is a carboxyvinyl polymer, from Noveon Inc.

Manufacturing Process:

1. Propylene glycol, sorbic acid, and dimethylisosorbide are mixed with ethanol and minoxidil is added to the mixture to produce a clear solution.

2. Carbopol 980 is added in small portions to the solution of step 1, with stirring.

3. Disodium EDTA and benzyl alcohol are dissolved in 90% of the water, and the solution is added to material of step 2 with vigorous stirring.

4. Cyclomethicone, dimethicone copolyol, and PPG-20 methyl glucan ether distearate are added to material of step 3.

5. Triethanolamine is dissolved in the remaining water and added slowly to the dispersion of step 5 with thorough mixing.

6. The stirring is continued overnight to allow the viscosity to stabilize and then the gel is filled into suitable containers.

### Example 9

### Spray Composition Containing Dutasteride

| Ingredient | Wt. Percent |
|---|---|
| Dutasteride | 0.5 |
| Dimethylisosorbide | 15 |
| Methyl paraben | 0.15 |
| Propyl paraben | 0.03 |
| Propylene glycol | 25 |
| Hydroxypropyl cellulose | 0.05 |
| Ethanol | 44 |
| Water | q.s. to 100 |

Manufacturing Process:

1. Dimethylisosorbide, propylene glycol, and ethanol are mixed together and dutasteride is added and dissolved.

2. Hydroxypropyl cellulose is dispersed in the drug solution with constant stirring.

3. Methyl and propyl parabens are dissolved in water.

4. The solution from step 3 is added to the polymer dispersion of step 2.

5. The viscosity is allowed to stabilize for 24 hours under slow stirring and the spray composition is filled into aerosol canisters, a propellant mixture of butane, isobutane and propane is added under pressure, in a ratio of 3 parts of propellant to 1 part of formulation, by weight, and aerosol valve closures are crimped onto the canisters.

### Example 10

### Foamable Composition Containing Dutasteride

| Ingredient | Wt. Percent |
|---|---|
| White petroleum | 1.25 |
| Cetearyl octanoate | 0.75 |
| Octyl dodecanol | 3 |
| Lecithin | 1.4 |
| Ceteareth 20* | 3 |
| Glyceryl stearate | 1 |
| Cyclomethicone | 1 |
| Methocel ™ K100M# | 0.3 |
| Xanthan gum | 0.25 |
| Water | 15 |
| Propylene glycol | 20 |
| Ethanol | 37.5 |
| Dimethylisosorbide | 15. |
| Phenoxyethanol | 0.5 |
| Potassium sorbate | 0.05 |

*Ceteareth 20 is a polyethylene glycol ether of cetearyl alcohol, used as an emulsifier.
#Methocel K100M is a special viscosity grade of hydroxypropyl methylcellulose supplied by Colorcon Asia Pvt. Ltd.

Manufacturing Process:

1. White petroleum, cetosteryl octanoate, octyl decanoate, lecithin, ceteareth 20, glyceryl stearate, and cyclomethicone are dissolved in a clean and dry vessel at 70° C.

2. Methocel K100M is mixed with water and heated to 60° C., and xanthan gum is added under vigorous stirring.

3. In another vessel, propylene glycol, ethanol, dimethylisosorbide, phenoxyethanol, and potassium sorbate are mixed thoroughly.

4. Dutasteride is added to the mixture of step 3 and stirring is continued to dissolve the drug completely.

5. The material of step 4 is added to the aqueous phase of step 2.

6. The material of step 5 is added to the oil phase of step 1 with stirring, to form an emulsion, and the emulsion is homogenized using a homogenizer.

7. The emulsion is filled into aerosol canisters; a propellant mixture of butane, isobutene and propane under pressure is added, in a ratio of 1 part propellant per 10 parts of emulsion, by weight, and aerosol valve closures are crimped onto the canisters.

### Example 11

### Microsphere-in-Gel Composition Containing Dutasteride

A. Microspheres:

| | |
|---|---|
| Ingredient | Quantity |

Aqueous Phase

| | | |
|---|---|---|
| Polyvinyl alcohol | 5 | g |
| Water | 1 | L |

Nonaqueous Phase

| | | |
|---|---|---|
| Ethylcellulose | 8.77 | g |
| Butylated hydroxytoluene | 0.18 | g |
| Isopropyl myristate | 0.88 | g |
| Dutasteride | 0.1 | g |
| Dichloromethane | 105 | mL |

Manufacturing Process:

1. Water is warmed to 80° C. and polyvinyl alcohol is added with stirring to form a vortex in the mixture to avoid clump formation.

2. The temperature is maintained at 60-80° C. for about 30 minutes, until polyvinyl alcohol dissolves completely.

3. The solution of step 3 is cooled to below 30° C. with stirring and filtered, then 585 mL of the filtrate is transferred into another vessel.

4. In another vessel, butylated hydroxytoluene, dutasteride and isopropyl myristate are mixed.

5. The mixture of step 4 is dissolved in dichloromethane with stirring.

6. Ethyl cellulose is added to the solution of step 5 in small portions, with stirring. During this step, clump formation and floating of polymer on the surface are avoided by forming a vortex. Stirring is continued for about 1 hour, until the polymer is dissolved completely.

7. The 585 mL of filtrate from step 3 is added to the solution of step 6, homogenized, and stirred overnight to allow the dichloromethane to evaporate.

8. The material of step 7 is filtered and the solid microspheres are washed with water and stored in the presence of activated silica gel for about 48 hours.

B. Gel:

Wt. Percent

| Ingredient | I | II | III | IV |
|---|---|---|---|---|
| Microspheres from A | 0.2 | 2 | 10 | 20 |
| Disodium EDTA | 0.05 | 0.05 | 0.05 | 0.05 |
| Propylene glycol | 5.5 | 5.5 | 5.5 | 5.5 |
| Sorbic acid | 0.1 | 0.1 | 0.1 | 0.1 |
| Glycerin | 5 | 5 | 5 | 5 |
| Carbopol 974P | 1 | 1 | 1 | 1 |
| Benzyl alcohol | 0.5 | 0.5 | 0.5 | 0.5 |
| Tween ™ 80 | 0.03 | 0.03 | 0.03 | 0.03 |
| Triethanolamine | 0.8 | 0.8 | 0.8 | 0.8 |

| | |
|---|---|
| Water | q.s. to 100 |

Manufacturing Process:

1. Disodium EDTA, propylene glycol, sorbic acid, glycerin, benzyl alcohol, and Tween™ 80 are dissolved in water.

2. Carbopol 974P is added to step 1 under continuous stirring while avoiding clump formation during, polymer addition.

3. The stirring is continued for approximately 2 hours, until the polymer is completely dispersed, to give a translucent smooth dispersion.

4. The dutasteride-containing microspheres are added to the material of step 3 under stirring, and the stirring is continued to obtain a homogenous dispersion.

5. Triethanolamine is diluted with a small quantity of water and a portion is added to the drug-polymer dispersion of step 4 under stirring,

6. The stirring is continued for 1 hour while monitoring pH, to reach and maintain about pH 4.5.

7. The addition of diluted triethanolamine is continued to obtain a pH 6 smooth gel.

8. The smooth gel obtained from step 7 is filled into collapsible tubes.

### Example 12

### Gel Composition Containing Dutasteride

| | Wt. Percent | | |
|---|---|---|---|
| Ingredient | I | II | III |
| Dimethylisosorbide | 15 | 15 | 15 |
| Propylene glycol | 20 | 20 | 20 |
| Ethanol, 96% | 30 | 30 | 30 |
| Butylated hydroxytoluene | 0.1 | 0.1 | 0.1 |
| Dutasteride | 0.5 | 0.5 | 0.5 |
| Transcutol | 5 | — | — |
| Propylene glycol | — | 5 | — |
| Oleic acid | — | — | 5 |
| Ethanol, 96% | 15 | 15 | 15 |
| Klucel HXF | 1.75 | 1.75 | 1.75 |
| Water | 12.65 | 12.65 | 12.65 |

Manufacturing Process:

1. Dimethylisosorbide, propylene glycol, ethanol (first quantity), and butylated hydroxytoluene are mixed to form “Phase A.”

2. In another container, dutasteride is mixed with Transcutol (in I), propylene glycol (in II), or oleic acid (in III), and ethanol (second quantity), to produce a clear solution “Phase B.”

3. Phase A is added to Phase B and stirred to obtain a clear solution.

4. Water is added to the solution of step 3 under stirring.

Klucel HXF is dispersed in the solution of step 4 under vigorous stirring and is allowed to swell completely under slow stirring.

5. The gel formed in step 5 is filled into a suitable container.

Gel compositions of Example 12 are stored at 40° C. and 75% RH for six months. Samples are analyzed before, during, and after storage, and the results are presented in Table 1. Impurities are expressed as percentages of the label dutasteride content and viscosity is expressed in units of poise, measured at 1000 rpm.

TABLE 1

| Composition | Parameter | Initial | 3 Months | 6 Months |
|---|---|---|---|---|
| I | Total Impurities | 0.11 | 0.14 | 0.07 |
| | Viscosity | 1.78 | 1.668 | 1.67 |
| | pH | 4.10 | 3.35 | 3.35 |
| II | Total Impurities | 0.16 | 0.19 | 0.1 |
| | Viscosity | 2.27 | 1.99 | 1.82 |
| | pH | 4.32 | 3.35 | 3.33 |
| III | Total Impurities | 0.09 | 0.18 | 0.08 |
| | Viscosity | 1.912 | 1.95 | 1.99 |
| | pH | 4.15 | 3.34 | 3.34 |

We claim:

1. A pharmaceutical composition for topical application, comprising a 5α-reductase inhibitor, or a pharmaceutically acceptable salt, ester, derivative thereof, and a pharmaceutically acceptable carrier, and optionally one or more other pharmaceutically acceptable excipients.

2. The pharmaceutical composition of claim 1, wherein a 5α-reductase inhibitor is a 5α-reductase type I inhibitor or 5α-reductase type II inhibitor.

3. The pharmaceutical composition of claim 1, wherein a 5α-reductase inhibitor comprises finasteride or dutasteride.

4. The pharmaceutical composition of claim 1, wherein a 5α-reductase inhibitor comprises dutasteride or a salt, ester, isomer, solvate, hydrate, or polymorphic form thereof.

5. The pharmaceutical composition of claim 1, wherein a pharmaceutically acceptable carrier is hydrophilic, hydrophobic, lipophilic, or ampiphilic, or a mixture thereof.

6. The pharmaceutical composition of claim 1, wherein a pharmaceutically acceptable carrier comprises a hydrophilic substance comprising an alcohol, a glycol, a cyclodextrin, or any two or more thereof.

7. The pharmaceutical composition of claim 1, wherein a pharmaceutically acceptable carrier comprises a hydrophobic or lipophilic substance comprising a paraffin oil, an ester of a C₈-C₁₈ organic acid, a C₈-C₃₀ fatty alcohol, a silicone oil, a vegetable oil, a fractionated or hydrogenated vegetable oil, a monoglyceride, a diglyceride, a triglyceride, a phospholipid, dimethylisosorbide, a volatile solvent, N-methylpyrrolidone, dimethylacetamide, dimethylformamide; dimethylsulphoxide, or any two or more thereof.

8. The pharmaceutical composition of claim 1, wherein one or more other pharmaceutically acceptable excipients comprise a surfactant, a co-surfactant, a penetration enhancer, an antioxidant, a buffering agent, a preservative, a viscosity modifying agent, a chelating or complexing agent, a coloring agent, a perfume, a polymer, a gelling agent, an alcohol, a liquid or semi-solid oily component, or any two or more thereof.

9. The pharmaceutical composition of claim 1, providing delivery of a 5α-reductase inhibitor onto the skin or scalp, beneath the skin or scalp, into systemic circulation, or any combination thereof.

10. The pharmaceutical composition of claim 1, providing topical delivery of a 5α-reductase inhibitor.

11. The pharmaceutical composition of claim 1, providing transdermal delivery of a 5α-reductase inhibitor.

12. The pharmaceutical composition of claim 1, which is permeation enhanced, penetration enhanced, or a depot composition.

13. The pharmaceutical composition of claim 1, comprising a penetration enhancer.

14. The pharmaceutical composition of claim 1, including a penetration enhancer comprising dimethylsulfoxide, decylmethylsulfoxide, diethylene glycol monoethyl ether, diethylene glycol monomethyl ether; 1-n-dodecylcyclazacycloheptan-2-one, propanol, octanol, benzyl alcohol, lauric acid, oleic acid, valeric acid, isopropyl myristate, isopropyl palmitate, methylpropionate, ethyl oleate; butanediol, polyethylene glycol monolaurate, urea, dimethylacetamide, dimethylformamide, 2-pyrrolidone, 1-methyl-2-pyrrolidone, ethanolamine, diethanolamine, triethanolamine, a terpene, a terpinoid, an alkanone, salicylic acid, a salicylate, citric acid, succinic acid, or any two or more thereof.

15. The pharmaceutical composition of claim 1, including an additional active agent comprising a hair loss preventing agent; a hair growth promoting agent, an anti-alopecia agent, an anti-infective, an antibacterial, an antifungal, an antihistaminic, an immunomodulatory agent, an anti-dandruff agent, an antiviral, an antiandrogenic agent, a hormone, a steroid, or any two or more thereof.

16. The pharmaceutical composition of claim 1, including an additional active agent comprising minoxidil.

17. The pharmaceutical composition of claim 1, comprising less than about 2 percent, based on the label content of 5α-reductase inhibitor, of total impurities having Formulae II-VI, after storage at 40° C. and 75% relative humidity for 6 months.

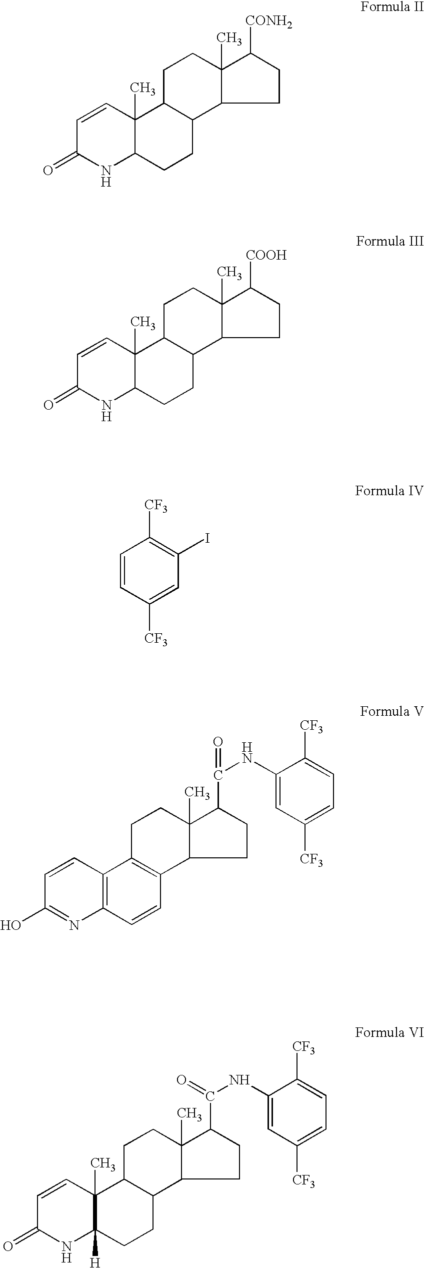

18. A process for preparing a pharmaceutical composition of claim 1, comprising: (a) dissolving or dispersing an active agent in a carrier, which is hydrophilic, hydrophobic or both; (b) adding a dermal penetration enhancer; (c) optionally, adding one or more surfactants; (d) optionally, adding one or more other pharmaceutically acceptable excipients; and (e) forming the mixture into a gel or cream.

19. A method of preventing or treating androgenic alopecia, comprising topically administering a pharmaceutical compositions of claim 1.

20. The method of claim 19, wherein a 5α-reductase inhibitor comprises dutasteride or a salt, ester, isomer, solvate, hydrate or polymorph thereof.
